(19) United States
(12) Patent Application Publication
(10) Pub. No.: US 2016/0296162 A1
(43) Pub. Date: Oct. 13, 2016

(54) DEVICE FOR SIGNALING MOVEMENTS OF A MUSCULOSKELETAL SYSTEM OF A HUMAN BEING

(71) Applicant: Jörg Alexander Böken, Sabershausen (DE)

(72) Inventor: Jörg Alexander Böken, Sabershausen (DE)

(21) Appl. No.: 15/021,610

(22) PCT Filed: Sep. 9, 2014
(86) PCT No.: PCT/EP2014/002431

§ 371 Date: Jul. 5, 2016

(30) Foreign Application Priority Data
Sep. 12, 2013 (DE) 10 2013 015 041.8

(51) Int. Cl. A61B 5/00; A61B 5/11

(52) U.S. Cl. CPC A61B 5/486; A61B 5/11; A61B 5/6804; A61B 5/7405; A61B 5/6846; A61B 5/7246; A61B 5/742; A61B 5/0022; A61B 5/746; A61B 2562/0247; A61B 2505/09; A61B 2560/0223

(57) ABSTRACT

The invention relates to a device for signalling movements of a musculoskeletal system of a human being having
a) at least one sensor (1) for detection of a movement,
b) at least one carrier element (2) for at least one sensor (1) for detection of a movement,
c) means (3) for fixing the at least one carrier element (2) to the musculoskeletal system of a human being,
d) a signalling device for signalling sensor values of the a east one sensor (1) for detection of a movement.

The invention relates to a device for signalling movements of a musculoskeletal system of a human being.

The support and musculoskeletal system of a human being ensures that the body remains in a specified form but nevertheless can be moved purposefully. The osseous skeleton is a part of the support and musculoskeletal system and ensures shaping of the body. The skeleton is moved by the skeletal muscles. For this, tendons which are attached to the bone on one side and are anchored in the muscle on the other side serve as power transmitters. If it becomes necessary to change the pulling direction of the tendons, they are deflected with ligaments. Such ligaments furthermore serve to strengthen and secure heavily loaded joints.

After accidents or injuries it may be necessary for the damaged part of the musculoskeletal system to be trained in a rehabilitation measure such that with correct guidance the musculoskeletal system to the greatest extent acquires its original mobility again, it being necessary for overloading to be avoided during this rehabilitation measure, so that the injury to the musculoskeletal system does not break out again.

It is known from the state of the art, for example, to employ orthotic devices, for example, to support and stabilise the musculoskeletal system in order to re-establish the original mobility of the musculoskeletal system and thereby, however, to avoid defective positions. Posture defects or position defects which are reversible, for example, can also be treated with such orthotic devices. There are furthermore also orthotic devices which are employed for temporary immobilisation of parts of the body.

It is to be said in principle that such orthotic devices restrict mobility considerably during the rehabilitation measure, so that the rehabilitation measure can be performed only in a very limited scope. Furthermore, by guiding the musculoskeletal system with such an orthotic device the user has only a limited capability of consciously approaching the limit of load tolerance in order thus to achieve an effective rehabilitation in a shortened window of time.

It is therefore the object of the invention to provide a device for signalling movements of a musculoskeletal system of a human being with which monitoring, control and also diagnosis of the musculoskeletal system in the orthopaedic sense exists for all the movable parts of the skeleton. With such a device posture damage in particular is to be prevented prophylactically or eliminated retrospectively, and movements or loads which cause damage to or overload the skeleton or the entire musculoskeletal system are to be avoided.

This object is achieved by a device having all the features of claim 1. Advantageous embodiments of the invention are to be found in the dependent claims.

The device according to the invention for signalling movements of a musculoskeletal system of a human being comprises at least one sensor for detection of a movement of the musculoskeletal system. This device furthermore has at least one carrier element for at least one sensor for detection of a movement of the musculoskeletal system, means for fixing the at least one carrier element to the musculoskeletal system of a human being also being provided. The device according to the invention moreover comprises a signalling device for signalling sensor values of the at least one sensor for detection of a movement of a musculoskeletal system of a human being.

With the aid of the device according to the invention, in the event of incorrect movements patients or users can be made capable, by a warning signal emitted by the device or the signalling device, of controlling and correcting themselves at any time in the event of a movement which is incorrect or overloads the musculoskeletal system. Such a warning signal is emitted, for example, when the at least one sensor for detection of a movement detects a movement which represents too high a load for the musculoskeletal system of the patient, which can possibly lead to an injury to the musculoskeletal system—and in a rehabilitation measure to a renewed breaking out of the injury.

Furthermore, patients with fractures which have already been treated by surgery can constantly monitor their momentary movements and thus make a valuable contribution to the healing process by avoiding an overload. The base settings of the device according to the invention and the adaptation to the patient or user is in each case to be made here by appropriate specialists, such as, for example, an orthopaedist, treating doctor, sports doctor, fitness trainer or the like. The user or the patient should furthermore be monitored by the particular specialist during an acclimatisation phase.

The device according to the invention can serve here in various possible embodiments, such as are also described below, for monitoring and diagnosis of axial movements, bending movements, rotary and torsion movements and for monitoring pressure, for example when treading too heavily with the foot or the like. Combination constructions with which the most diverse movements can be monitored and controlled in combination and with which a warning signal can be emitted in the event of overloading of the musculoskeletal system are furthermore also possible. By this means the user or the patient is made capable of adapting the movement accordingly if such a warning signal occurs. With the device according to the invention a large number of combination constructions can be realised for this in the nature of a modular system in order to be able to monitor and control a large number of movements of the musculoskeletal system.

In rehabilitation measures in particular, the musculoskeletal system can be trained again with a device according to the invention such that after adaptation of the device by the specialist the patient can train and consequently control the musculoskeletal system again accordingly without running the risk of overloading the musculoskeletal system, so that a renewed injury is effectively avoided.

According to a first advantageous embodiment of the invention the at least one sensor is constructed as a distance sensor, pressure sensor, rotary movement sensor, linear movement sensor, torsion sensor, extension and/or position sensor. With the aid of such sensors it is possible to monitor virtually all the movements of the musculoskeletal system of a human being. In particular, bending and rotary movements as well as stretching movements and also pressure loads of the musculoskeletal system can be effectively detected by this means. In this context a maximum value which is not to be exceeded is assigned to the particular sensor by the specialist after adaptation, in order to avoid renewed and/or new injuries to the musculoskeletal system. If the signal value of the sensor approaches this maximum value during a movement an appropriate warning signal which can be perceived by the user/patient is generated by the signalling device before or when the maximum value is reached. Both simple mechanical switch contacts and more complicated movement and pressure sensors are sensors which can be used in this context. In particular, mechanical contacts, such as, for example, microswitches and microbuttons, but also optoelectronic components, such as, for example, PIR sensors, light barriers, fork light barriers, lasers, any type of pressure sensors, and also magnet field sensors, such as, for example. Hall effect transducers, Hall effect sensors or reed switches, inductive, capacitive and infrared as well as ultrasound proximity sensors and angle sensors of any construction as well as radar sensors can be employed as sensors. With the aid of such sensors and an appropriate evaluation logic it is possible, for example, to convert distance values into pressure values and vice versa.

According to a further concept of the invention it has proved appropriate for the at least one carrier element to be constructed as at least two flexible plates connected via bars, preferably at least one sensor being arranged between two bars. With the aid of such flexible plates it is possible to detect in particular bending movements of a joint and/or also pressure loads between the plates and therefore pressure loads on the particular joint. From the values for these sensors conclusions can thus be drawn regarding the load on the musculoskeletal system in the particular region at which the device according to the invention is arranged on the musculoskeletal system of a human being.

Alternatively, it can also be envisaged that the at least one carrier element is constructed as at least two member elements connected via a joint, wherein preferably at least one sensor is arranged between two member elements. The joint can be constructed here for articulated connection of the member elements in one plane. However, it is also conceivable that the joint connects the two member elements with one another in an articulated manner in any spatial direction.

According to another concept of the invention, however, it is also possible for the at least one carrier element to be constructed as a flexible traction cord, band or the like, for example in the form of a collar. Such carrier elements are suitable in particular for detecting movements in the range of a limb, such as, for example, a leg or an arm, or also, as a neck collar, the movement of the musculoskeletal system in the region of the cervical vertebral column, with the aid of the particular sensors or a sensor.

With the last two carrier elements mentioned it is also possible to draw from the values of the sensors conclusions regarding the load on the musculoskeletal system in the particular region at which the device according to the invention is arranged on the musculoskeletal system of a human being.

So that the device according to the invention can be fixed to the musculoskeletal system of a human being, means for fixing are provided, which are constructed, for example, as self-adhesive pads, bandages, plaster, collars, splints, adhesive plasters, hook and loop fasteners, items of clothing and/or implants or the like. With the aid of these fixing means it is possible in a simple manner for virtually all intended uses to fix the device according to the invention or carrier elements thereof with the sensors to the musculoskeletal system of a human being, so that the movement in the particular desired region of the musculoskeletal system of a human being can be monitored, controlled and if appropriate also diagnosed in a simple manner, it being possible for defective movements which can lead to damage to the musculoskeletal system to be signalled early on by the signalling apparatus with the aid of the sensor values.

It has furthermore proved to be particularly advantageous here for the signalling device to be constructed as a data processing device. With the aid of such a data processing device it is possible in a simple manner to evaluate the data supplied by the sensors and to allow a signalling to take place as required. Such a signalling can take place, for example, acoustically or also via a light signal. However, it is also conceivable to display the movement of the musculoskeletal system at least in the region of the device according to the invention or of the particular carrier element visually via a data output unit, for example a screen, so that the movement or the defective movement in the case of a warning signal can be immediately visualised to the user visually, for example on the screen of a smartphone or the like. An immediate training and/or learning effect can be achieved particularly rapidly and efficiently by this means.

It is furthermore also possible for the signalling device or the data processing device to have a data storage unit in which the sensor data can be stored. Reference data for the particular sensors can moreover also be filed in such a data storage unit, so that the data supplied by the sensors can be compared with the particular reference data and if the reference signals are deviated from or exceeded an appropriate warning signalling can be issued.

The sensor values recorded by the device according to the invention or sensors thereof can be transmitted with the aid of an appropriate electronic system to an evaluation unit, no that an appropriate data output can then take place on the particular data output apparatus. In the case of a visual presentation of the data output on a screen in particular, it is possible for the user/patient to see not only that he has made an error but also directly what error he has made. In this respect this embodiment of the invention is particularly effective in particular in the educational respect, since this can be corrected directly by the user immediately, via a visual perception of the error, so that rapid and efficient practising can take place.

For the data processing, an evaluation device is also provided, with the aid of which the data recorded by the sensors can be compared with the reference data and differences in the course of the movement which have been recorded with the aid of the sensors can thus also be compared with a course of the movement which has been optimised by the reference data. The patient is thus made capable of practising the desired course of the movement, during which he can follow his course of the movement, for example, directly on a monitor and can compare it with an optimum course of the movement.

Wireless connections, such as, for example, radio transmitters, WLAN, infrared or Bluetooth, can be provided for transmitting the data to the evaluation unit or the signalling device. Nevertheless, transmission can also take place through appropriate wiring. With the aid of a wireless connection, however, the data recorded by the sensors can be passed on directly to the evaluation unit or signalling device or data processing without cumbersome wiring. With the aid of the data processing device and the corresponding sensors, long-term data acquisition and evaluation is possible. With the aid of the data processing unit and corresponding computer programs or applications, the entire data acquisition can also be presented in three-dimensional graphics. Furthermore, computer-animated courses of the movement can also be reconstructed over longer phases.

The entire calibration of the sensor system can be realised both mechanically and electronically, depending on the construction and type of the sensors used. Nevertheless, it is to be ensured here that the calibration is carried out by specialist persons such as, for example, orthopaedists, doctors, sports doctors or trained fitness trainers, in order to achieve an optimum action.

With the aid of the device according to the invention monitoring and diagnosis of the complete or partial skeleton and musculoskeletal system for loading and overloading of the musculoskeletal system is possible.

Fields of use are, for example, clinical use on injuries following an operation or in the event of a chronic disorder of the skeleton. The device according to the invention can furthermore also undergo an orthopaedic use for recognition and long-term treatment of posture damage. Such a device according to the invention can also be used prophylactically for early prevention of posture and movement damage, in particular in human beings with a sedentary occupation.

It is of course also possible to employ the device according to the invention orthopaedically in competitive and popular sport.

Further aims, advantages, features and possible uses of the present invention emerge from the following description of examples with the aid of the drawings. In this context, all the features described and/or shown in the figures, by themselves or in any desired appropriate combination, form the subject matter of the present invention, also independently of their combination in the claims or their references.

The figures show:

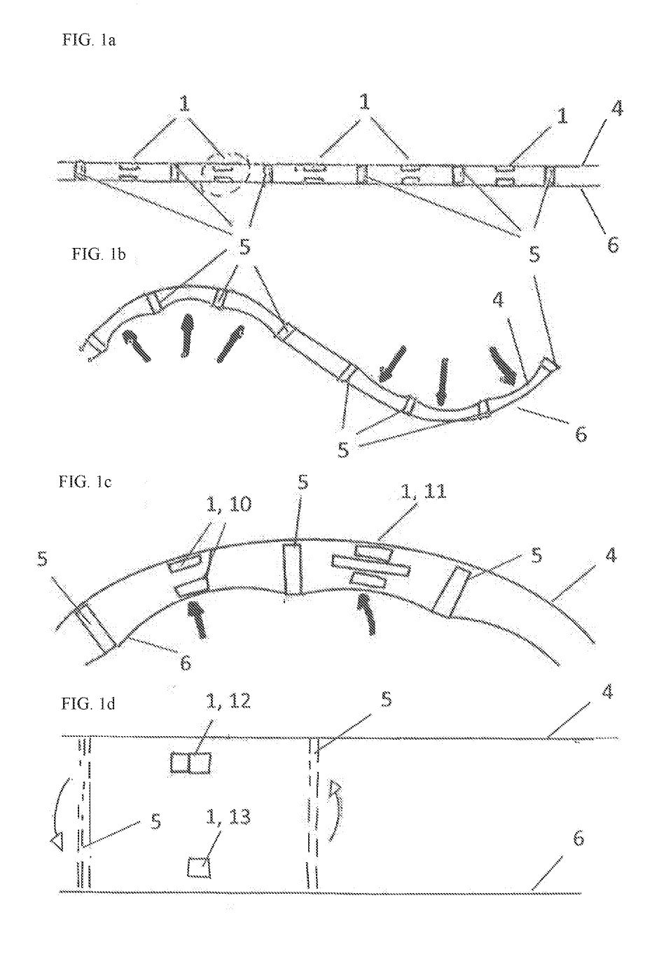

FIGS. 1a to d: an example of a device according to the invention in various representations,

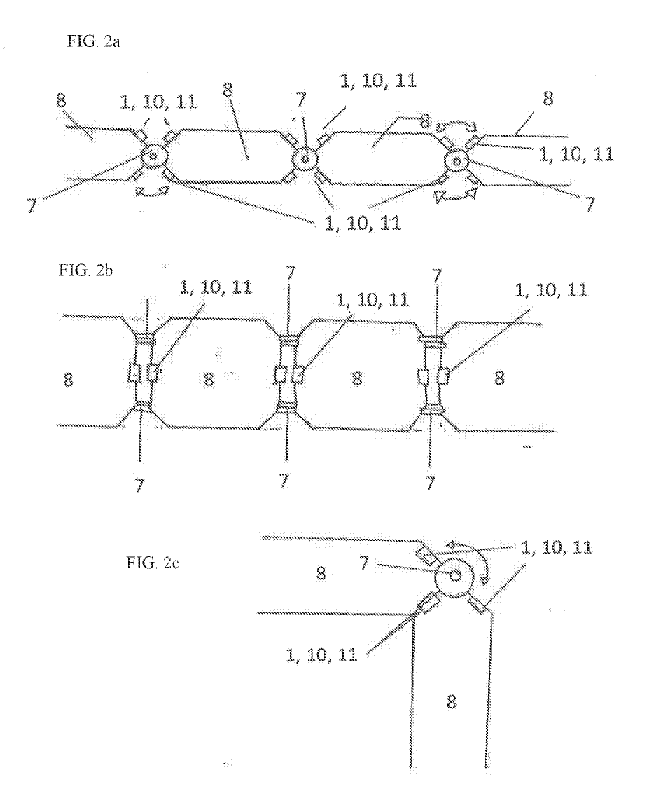

FIGS. 2a to c: a second example of a device according to the invention in various representations,

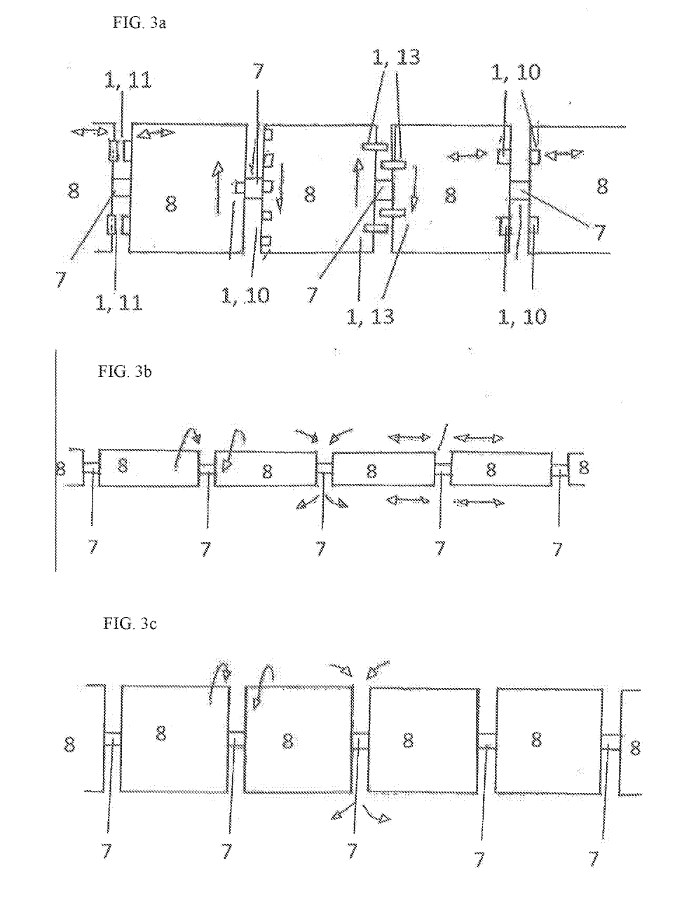

FIGS. 3a to c: a third example of a device according to the invention,

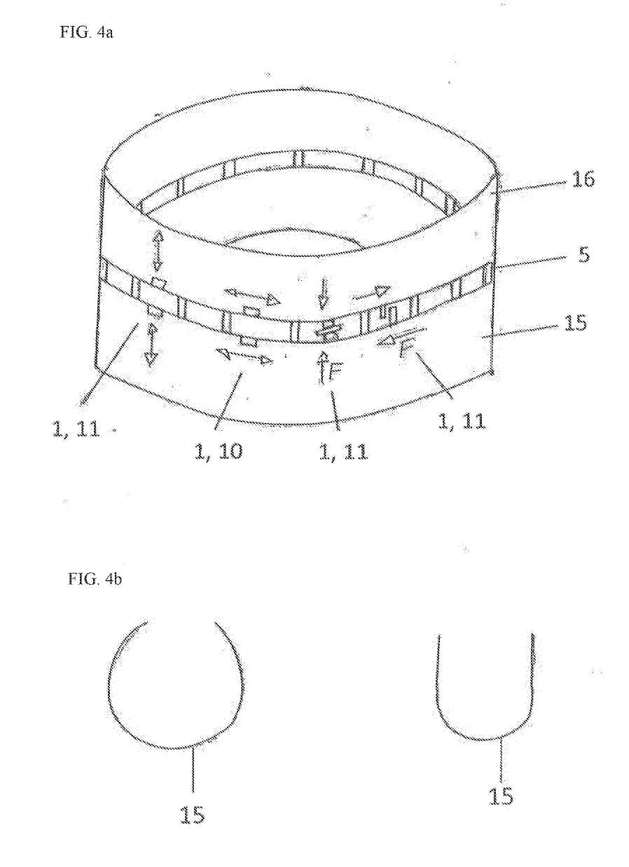

FIGS. 4a to c: a fourth example of a device according to the invention in various embodiments,

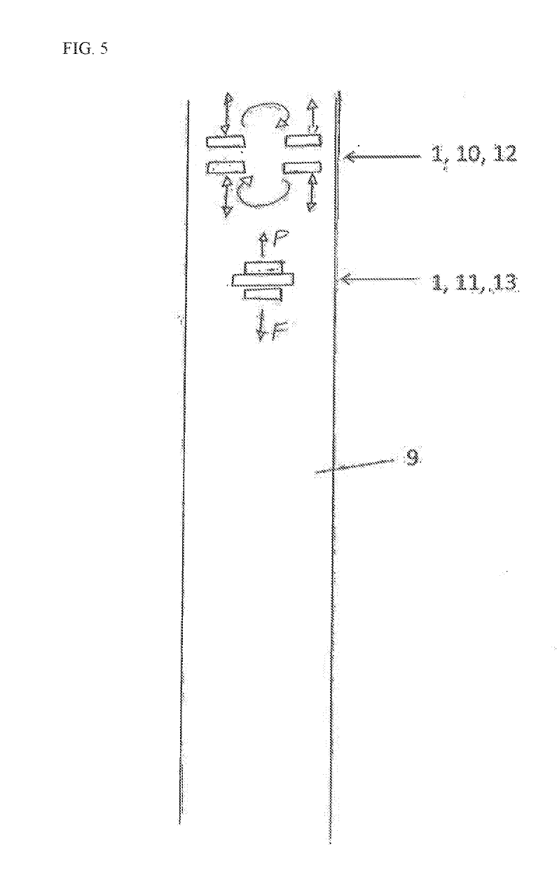

FIG. 5: a fifth example of a device according to the invention,

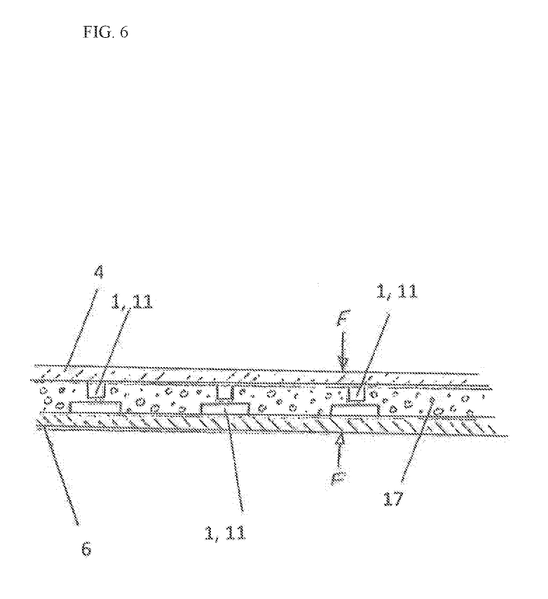

FIG. 6: a sixth example of a device according to the invention,

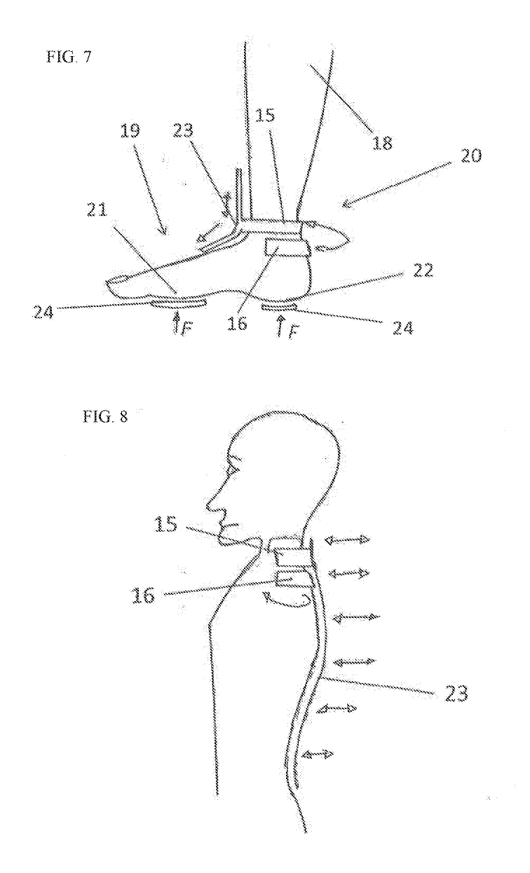

FIG. 7: a representation of the use of various devices according to FIGS. 1 to 6 on a leg or foot and ankle of a human being and

FIG. 8: a representation of the use of a device according to the invention in the region of the vertebral column and the cervical vertebral region of a human being.

FIG. 1a shows a first example of a device according to the invention for signalling movements of a musculoskeletal system of a human being. This example substantially comprises an upper flexible plate 4 and a lower flexible plate 6 which are connected to one another by means of fixed bars 5. Between the upper flexible plate 4 and the lower flexible plate 6 sensors 1 for recording movements are furthermore arranged between the individual bars 5. In the representation of FIG. 1a the device according to the invention there is arranged with its upper flexible plate 4 and lower flexible plate 6 in linear form.

As can be seen from FIG. 1b, the flexible plates 4 and 6 are bendable against one another in the plane of the drawing there. As a result of the plates 4 and 6 being kept at a distance by the bars 5 which are fixed here, on bending of the plates 4 and 6 the bend radius, which is shown with arrows in this representation, changes. Due to the flexibility of the plates 4 and 6, as a result the distance between the plates 4 and 6 becomes smaller or the pressure between the plates 4 and 6 becomes greater on bending. For reasons of clarity, however, the individual sensors 1 are not shown in the representation according to FIG. 1b.

The representation of FIG. 1c, however, shows the detailed representation of a bending according to FIG. 1b, wherein on the one hand the distance between the plates 4 and 6 can be measured with a sensor constructed as a distance sensor 10 and on the other hand the pressure between the plates 4 and 6 can be determined with a sensor 1 constructed as a pressure sensor 11.

FIG. 1d furthermore gives a plan view of another region of this device according to the invention. In this, a position sensor 12 and a torsion sensor 13 are now used as sensors 1. By means of the torsion sensor 13 the torsion between two bars 5 can be measured, while by means of the position sensor 12 the position of this sensor can be determined.

The sensor values can all be processed by means of a signalling device, which is not shown in the representations according to FIGS. 1a to 1d—or also in all further following representations—and which is preferably constructed as a data processing device. In this context, with a corresponding logic filed in a data storage unit of the data processing device it is also possible to convert the individual sensor values into other physical parameters, for example a pressure into a distance value or vice versa, distance value being understood as meaning both length distances and angle distances.

The data processing device preferably comprises a data storage unit, a data reading unit, a data output unit and an evaluation device. By means of the data reading unit the sensor data of the individual sensors 1, 10, 11, 12 and 13 can be read and stored in a data storage unit or processed or evaluated directly by the evaluation device.

By means of a data output unit a very exact data output can take place, which renders possible, for example, a visualisation of the movement on a screen. With the aid of the evaluation device the sensor data recorded can be compared with reference data stored in the data storage unit, so that if a reference value is exceeded or reached by a sensor a warning signal can be issued. In the simplest embodiment such a warning is an acoustic signal. Needless to say, complex courses of movement from a large number of the most diverse sensors can also be visualised with the aid of the data processing device and displayed on a corresponding presentation apparatus. In this context the most diverse warnings can be generated when different sensors exceed the reference values corresponding to a preset entry. With such a visual two- or three-dimensional presentation courses of movement can also be matched in a simple manner to optimised courses of movement which are stored in the data storage unit. The patient can detect directly a difference between a completed, defective and an optimised movement optically on a screen and in response immediately take counter-measures in order the correct the defective movement and to train the optimised course of the movement.

Preferably, the transmission of the sensor data is wireless, for example by means of a radio module. However, it is of course also possible to pass on the data to the data processing device via wiring.

To record the movement data of a musculoskeletal system of a human being, for this the flexible double strip comprising the flexible plates 4 and 6 connected by the bars 5 is arranged on the region of the musculoskeletal system of a human being which is to be monitored. Fixing can be effected here in various ways and manners, for example by self-adhesive pads, by bandages, by plaster, by collars, by splints, by sticking plasters, by hook and loop fasteners or also by items of clothing into which such a double strip can be integrated. In the case of such items of clothing, however, these must lie closely on the body or the musculoskeletal system; for example, these can be a close-fitting T-shirt, cycling trousers, a neoprene suit or similar items of clothing.

FIGS. 2a to 2c show a second example of a device according to the invention. This substantially comprises several member elements 8 connected to one another in an articulated manner via a joint 7, in each case at least one sensor 1 being arranged between the member elements 8.

FIG. 2a gives a plan view of this example, it being possible for the individual members 8 to be swivelled relative to one another by means of the particular joint 7 only in the plane of the drawing. FIG. 2a shows here this example in a linear arrangement.

FIG. 2b shows a side view of the representation according to FIG. 2a. The sensors 1 here are constructed as distance sensors 10 and pressure sensors 11, so that with the aid of these sensors an angle with which adjacent members 8 can he swivelled relative to one another can be determined. In the representation according to FIG. 2b the individual members 8 can be swivelled relative to one another out of the plane of the drawing.

FIG. 2c shows the example of FIGS. 2a and 2b in a representation in which two adjacent members 8 are swivelled relative to one another by 90°. In this context the one distance sensor 10 detects the maximum angle of opening, namely 90°, while the other distance sensor 10 detects a value of 0. However, with these members 8 swivelled relative to one another by 90°, at the minimum distance of 0 the members are directly adjacent to one another so that the pressure with which the members 8 press against one another can be measured there by means of the pressure sensor 11.

FIGS. 3a to 3c show a third example of a device according to the invention. This example substantially corresponds to that of FIGS. 2a to 2c, the joints 7 here being constructed for the members 8 to swivel relative to one another not only in one plane. Rather, adjacent members 8 can also swivel spatially relative to one another by means of the joints 7. Regarding now FIG. 3a, various sensors 1 for various states of movement and load are arranged in the device according to the invention there. The sensor 1 on the far left in this representation is constructed here as a pressure sensor 11 which can record pressures between the two members 8 on the left and pass them on to the data processing device. The second sensor 1 from the left is now constructed as a distance sensor which, however, detects not length distances but rotational distances in the form of angles. The third sensor 1 from the left is constructed as a torsion sensor, with the aid of which the torsion between the third member 8 from the left and the fourth member 8 from the left can be measured. The sensor 1 on the right is in turn constructed as a distance sensor, it now being possible to record an axial shift between the member 8 on the right and the second member 8 from the right.

FIG. 4a shows a collar arrangement which comprises an upper collar 16 and a lower collar 15 which are connected to one another by means of bars 5. Various sensors 1 are moreover arranged between the collars 15 and 16. With the aid of the sensor on the left, which is constructed as a distance sensor, an inclination between the upper collar 16 and the lower collar 15 can be detected. A second distance sensor now detects an angle with which the upper collar 16 is rotated relative to the lower collar 15. The third sensor 1, which is shown as the second from the right in this representation, is now again constructed as a pressure sensor 11 and serves to determine the axial force of inclination or the inclination pressure with which the upper collar 16 is swivelled relative to the lower collar 15. The sensor 1 on the right is likewise constructed as a pressure sensor 11, but here it is not the inclination of the collars 15 and 16 relative to one another which is determined, but the rotation thereof, namely the torsion.

FIGS. 4b and 4c show further embodiments of collar-like devices according to the invention, here, however, only the form thereof being indicated and the placing of the individual sensors 1 not being shown.

In the example according to FIG. 5 various sensors are arranged on a flexible tape 9. This tape can be made, for example, of an elastic plastic or of a rubber tape or also of a silicone strip.

On this flexible tape 9 a sensor 1, which is constructed as a distance sensor 10 and position sensor 12, is arranged in the upper region. With the aid of this sensor 1, for example, an extension of the tape 9 and also a torsion of the tape 9 can be detected and transmitted further to the data processing unit. The lower sensor 1 here is constructed as a pressure sensor 11 or torsion sensor 13, with the aid of which the force or the pressure with which the flexible tape is extended or the torsion with which the flexible tape 9 is twisted can be determined. Since the flexible tape is arranged directly on the musculoskeletal system of a human being, by the movements of the flexible tape which are obtained through the sensor signals conclusions regarding movements of the musculoskeletal system or regarding loads on the musculoskeletal system can be drawn directly.

FIG. 6 now shows an example of a device according to the invention in the form of a plate construction. Here also this plate construction comprises an upper plate 4 and a lower plate 6, between which here, however, an intermediate layer 17 serving, for example, for damping is arranged. In this example the sensors 1, which are constructed here as pressure sensors 11, are now arranged within this intermediate layer 17. With the aid of these pressure sensors pressure loads, for example when trod on with a foot 19, can be determined.

FIGS. 7 and 8 now show the most diverse cases of how a device according to the invention can be used. In this context FIG. 7 shows a lower leg 18 with the associated foot 19 and connecting ankle 20. In the region of the ball of the foot 21 and the heel 22 devices 24 according to the invention according to the representation of FIG. 6 are arranged here, with the aid of which the pressure with which the foot 19 or the ball of the foot 21 and the heel 22 are loaded when treading can be measured.

Furthermore, a device according to the invention with a double function is arranged in the region of the ankle. On the one hand this device comprises a device 23 according to the representations in FIG. 1, 2, 3 or 5, with the aid of which the angle of inclination and the inclination pressure between the lower leg 18 and foot 19 can be determined. Furthermore, an upper collar 15 and a lower collar 16 are used here, with the aid of which a rotation of the foot 19 relative to the lower leg 18 can be detected. Both the angle with which the foot 19 is rotated relative to the lower leg 18 and the torsion during such a rotation can be detected here.

Finally, FIG. 8 shows a device according to the invention for monitoring and control of the movement of a vertebral column and the cervical vertebral region of a human being. For this, a device 23 according to the representations in FIGS. 1, 2, 3 or 5 is used for monitoring or control of the movement of the vertebral column. In this context a collar-like device is used for monitoring and control of the cervical vertebral region, an appropriate sensor system for recording distances and pressure or forces being arranged between an upper collar 15 and a lower collar 16.

For all the embodiments shown here in the figures there is a wide selection with respect to the choice of the material of the carrier element 2. The selection of the material is made here taking into account the particular aim and purpose of monitoring or control of the individual regions of the musculoskeletal system. In principle metals, plastics in the form of thermosets or thermoplastics or the like, as well as all textiles as synthetic and/or natural fibres and also other products of rubber, elastic rubber or the like as well as wood and other natural products can be used for this.

### List of Reference Symbols

1 Sensor
2 Carrier element
4 Plate
5 Bar
6 Plate
7 Joint
8 Member element
9 Tape
10 Distance sensor
11 Pressure sensor
12 Position sensor
13 Torsion sensor
14 Inclination sensor
15 Collar
16 Collar
17 Intermediate layer
18 Leg
19 Foot
20 Ankle
21 Ball of the foot
22 Heel
23 Device according to FIG. 1, 2, 3 or 5
24 Device according to FIG. 6

1. Device for signalling movements of a musculoskeletal system of a human being having
a) at least one sensor (1) for detection of a movement,
b) at least one carrier element (2) for at least one sensor (1) for detection of a movement,
c) means for fixing the at least one carrier element (2) to the musculoskeletal system of a human being,
d) a signalling device for signalling sensor values of the at least one sensor (1) for detection of a movement, wherein the at least one carrier element (2) is constructed as at least two member elements (8) connected via a joint (7), wherein at least one sensor (1) is arranged between two member elements (8), wherein the sensors (1) are constructed as distance sensors and pressure sensors, so that an angle can be determined with the aid of these sensors (1).

2. Device according to claim 1, characterised in that the sensor (1) is constructed as a distance sensor, pressure sensor, rotary movement sensor, linear movement sensor, torsion sensor, extension sensor and/or position sensor.

3. Device according to claim 1 or 2, characterised in that the at least one carrier element (2) is constructed as at least two flexible plates (4, 6) connected via bars (5), wherein preferably at least one sensor (1) is arranged between two bars (5).

4. (canceled)

5. Device according to one of the preceding claims, characterised in that the at least one carrier element (2) is constructed as a flexible traction cord, flexible tape (9) or the like, for example in the form of a collar.

6. Device according to one of the preceding claims, characterised in that the means (3) for fixing are constructed as self-adhesive pads, bandages, plaster, collars, splints, sticking plasters, hook and loop fasteners, items of clothing and/or implants.

7. Device according to one of the preceding claims, characterised in that the signalling device (1) is constructed as a data processing device.

8. Device according to one of the preceding claims, characterised in that the signalling device (1) comprises a data storage unit, a data reading unit, a data output unit and an evaluation unit.

9. Device according to claim 8, characterised in that the data storage unit is constructed for storage of sensor signals, which preferably can be compared by the evaluation unit with reference values for sensor signals filed on the data storage unit or a data storage unit remote from the device.

10. Device according to one of the preceding claims, characterised in that the signalling device (1) is constructed for battery or accumulator operation.

11. Device according to one of the preceding claims, characterised in that the signalling device (1) is constructed as a portable apparatus which is preferably constructed for acoustic and/or visual signalling.

12. Device according to one of the preceding claims, characterised in that it or the at least one sensor (1) is constructed for wireless transmission of sensor signals, preferably to the signalling device.
